(19) United States
(12) Patent Application Publication
(10) Pub. No.: US 2003/0124199 A1
(43) Pub. Date: Jul. 3, 2003

(54) Use of fly larval extracts for wound treatment

(76) Inventors: Karl-Heinz Nietsch, Neuss (DE); Rainer Pooth, Dreieich-Gotzenhain (DE); Heinz Mehlhorn, Neuss (DE); Thomas Ruzicka, Dusseldorf (DE); Helger Stege, Freiburg (DE)

Correspondence Address: ROSS J. OEHLER, AVENTIS PHARMACEUTICALS INC., ROUTE 202-206, MAIL CODE: D303A, BRIDGEWATER, NJ, 08807, US

(21) Appl. No.: 10/215,593

(22) Filed: Aug. 9, 2002

Related U.S. Application Data
(60) Provisional application No. 60/349,012, filed on Jan. 14, 2002.

(30) Foreign Application Priority Data
Aug. 10, 2001 (DE) 10138303.7
Oct. 4, 2001 (DE) 10149153.0

(51) Int. Cl. A61K 35/64

(52) U.S. Cl. 424/538

(57) ABSTRACT

The invention relates to the topical application of fly larval extracts obtainable from fly larvae which are killed and extracted with cooling in aqueous medium or in solvents and are freed of undissolved constituents. The fly larval extracts of various species are suitable for the treatment of superficial or deep chronic and acute wounds of any etiology. The fly larval extracts with a wound-healing effect are obtainable for example from fly larvae of the genera Sarcophaga or Lucilia.

### CROSS-REFERENCE TO RELATED APPLICATIONS

This application claims priority from U.S. Provisional Application No. 60/349,012 filed Jan. 14, 2002, incorporated herein by reference.

### BACKGROUND OF THE INVENTION

The invention relates to the topical application of fly larval extracts of various species which belong, for example, to the genera Sarcophaga or Lucilia for the treatment of superficial or deep chronic and acute wounds of any etiology.

Wound healing is a complex event in which multiple target cells and target structures must intermesh in an ordered sequence of processes. These processes proceed irrespective of the type of wound (chronic/acute), while the duration of individual phases is variable. It is possible in general to distinguish three main phases, the exudative phase, the proliferative phase and the epithelialization or repair phase. In the exudative phase, the acute traumatization of the hemostatic and vasoconstricting reactions predominates. The main clinical features are edema of the wound and pain from the wound. The resulting vascular defect is closed under the influence of the platelets. These release substances with chemotactic activity. Macrophages, neutrophils and lymphocytes migrate in. This sets up a phagocytosis system which is highly effective for tissue debridement.

After elimination of cell detritus, the proliferative phase begins with the migration of fibroblasts and vascular endothelial cells. There is a massive increase in the cell content due to the migration in of fibroblasts and endothelial cells. At the same time, there is increased release of cytokines and growth factors which in turn stimulate formation of new vessels and cellular proliferation. In addition, matrix transformation takes place in this phase. This occurs due to the formation and transformation of type III collagen into type I collagen. The result is a granulation tissue with good capillarity and rich in macrophages, fibroblasts and mast cells.

This proliferative phase is followed by the epithelialization and repair phase. In this final phase there is contraction of the wound and migration of marginal keratinocytes into the wound. There is a decrease in neoangiogenesis and capillary density. By contrast, there is an increase in the collagen content. This process is relevant for the mechanical strength of the resulting scar tissue.

If these complex interactions are impaired there may be delayed wound healing. Depending on the cause of the impaired processes there are said to be chronic disturbances of wound healing after a wound has existed for 6-8 weeks. These occur with a large number of immunological disorders, with varicosis, with arterial occlusive disease, after infections and, for example, in diabetes mellitus. Measures to promote wound healing serve to expedite or regularize the sequence of the processes described above. The methods which should be mentioned in this connection are primarily those for cleaning wounds, in addition to those promoting granulation. However, epithelialization can also be promoted by modern wound dressings. One of the main reasons for delayed wound healing is insufficient formation of granulation tissue. This may be caused by diminished endogenous wound debridement, or infections, disturbances of blood flow, immunological disorders result in an excessive formation of cellular and tissue detritus.

Measures for wound cleaning are employed therapeutically in these cases. The aim of the cleaning is produce a clean wound bed. For this purpose it is first necessary to eliminate residues of ointment and crusts and possibly cut away necroses which are present. The latter takes place surgically with a sharp spoon (curettage) and with forceps and scissors. An alternative possibility is to use enzymatic ointments which preferably degrade denatured protein. They contain enzymes such as trypsin, chymotrypsin, enzymes from bovine material such as pancreatic enzymes, or collagenase, fibrolysin, streptokinase or calves' blood dialysates. In parallel with this, regular disinfection takes place for example with potassium permanganate or with Rivanol® baths. Disinfecting measures can also be carried out with silver- or iodine-containing preparations.

However, enzymatic products often show only limited efficacy on the patient. This is because the dosage of the enzyme is often very low and the half-life of the known enzyme products is 6 to 12 hours. This is why dressings must be changed each day, even several times. Some of the products are combination products with antibiotics which can be applied topically. The disadvantage of the combination products is the risk of epicutaneous sensitization. Almost 60-70% of patients with chronic leg ulcers suffer from one or more sensitizations to ointment bases or other constituents of topical preparations. The use of local antibiotics should, therefore, be dispensed with because, on the one hand, the development of resistance and, on the other hand, the sensitization rate is high.

It is also known to place larvae (maggots) of the species Lucilia sericata on wounds. This therapy is based on age-old folk remedies and partly on findings and observations by military medical officers about the contamination of war wounds with fly maggots. The maggots of this species feed exclusively on necrotic tissue. This involves this material being almost predigested by secretion of saliva and only then being taken in by the maggot. There is never an active intake of food by maggots of L. sericata in the sense of a chewing or biting action. This ensures that the maggots cannot penetrate into other regions of the body or into unaffected body cavities. The maggot therapy shows very high therapeutic efficacy. However, the current treatment method is extremely complicated, costly and requires great logistical effort. Maggots for use on humans must be grown under controlled conditions. Sterility must be ensured both for the rearing and for the transport from the laboratory to the patient. The therapeutic efficacy cannot be metered. Although determination of the number of maggots applied to the wound is possible, that of the enzymatic activity afforded by them is not. The maggots themselves are subject to a process of biological development ending in metamorphosis of the larva to the pupa and then to the fly. For this reason, application of the larvae must be repeated at short intervals. A high degree of compliance is necessary for patients and providers of medical services in order to carry out the therapy because it is necessary psychologically to cross cultural and civilizational boundaries. In addition, the insertion of the larval mouth hooks may be quite painful.

The patent application WO 01/31033 described a protein which is secreted to the outside by live maggots of the species Lucilia sericata and which is assumed to have a wound-healing property. However, physiological experiments on wounds which might support this supposition are lacking. The application WO 01/31033 describes the isolation of a very small amount of the secreted protein, but an economic process for obtaining marketable quantities of protein would need to be developed. Also known is an oily formulation of a powder of dried fly larvae in oil which is or has been employed for wound treatment in China (Yang Zheng.: China-Science: House fly yields medicine, expert say. In: Inter Press Service; 03.09.1997, 97:314231 NLDB). A disadvantage of the oil is that it may cause allergies as a side effect. The oily formulation of the Chinese product is contrary to the state of the art of wound treatment in Western medicine: the current and general view is that modern wound dressings should have a hydrophilic milieu, with which better healing is observed than on use of wound-covering hydrophobic formulations (Pontieri-Lewis V. (1999) Principles for selecting the right wound dressing. Medsurg Nurs 8:267-70; Casey G (2001) Wound dressings. Paediatr Nurs 13:39-42; Casey G (2000) Modern wound dressings. Nurs Stand 15:47-51; Ruszczak Z, Schwarz R A (2000) Modern aspects of wound healing: an update. Dermatol Surg 26:219-229; Probst W (2000) Lokale Behandlung chronischer Wunden. Pharm Ztg 145:3907-3920; Strobel H-G (2000) Wundfibel; Qualitätsstandards zur Wundbehandlung an der Universitätsklinik Essen. Krankenhauspharmazie 21:350-361). The disadvantages of oily formulations are, in particular, that they inhibit the migration and function of immune cells and interfere with the proliferation of newly forming cells.

### SUMMARY OF THE INVENTION

In the endeavor to find effective methods of treatment of superficial, deep, chronic or acute wounds of any etiology, it has now been found that the extracts of the invention from fresh fly larvae of various species, for example of the genera Sarcophaga or Lucilia, are able to eliminate the disadvantages mentioned.

The fly larval extracts of the invention represent a marked further improvement in maggot therapy in relation to application and dosage. The extracts can also be used in modern hydrophilic wound dressings. Standardization of the method of manufacture makes it possible to control the therapy better. As a finished product, the fly larval extracts have a continuous efficiency which does not depend on the maggots' development cycle.

The invention therefore relates to fly larval extracts obtainable from fly larvae, where the fly larvae are first cooled and then homogenized, and the resulting homogenate is finally freed of undissolved constituents of the fly larvae.

It is possible where appropriate to add an extraction medium before the homogenization. The extraction medium contains water or is an organic solvent. The soluble constituents may moreover be preserved or immediately applied topically to the wounds. The extract of the invention has on topical application a wound-healing effect on superficial, deep, chronic or acute wounds of any etiology.

### DETAILED DESCRIPTION

Suitable fly larvae are derived for example from the genera Sarcophaga, Lucilia, Musca, Calliphora and Stomoxys. It is also possible to employ mixtures of fly larvae from said genera in the method of the invention. Suitable species from said genera are, for example, Lucilia sericata, Lucilia caesar, Lucilia cuprina, Sarcophaga carnaria, Sarcophaga agyrostoma, Musca domestica, Calliphora erythrocephala, Calliphora vicina or Stomoxys calcitrans. The genera Sarcophaga and Lucilia for example are ubiquitous and a skilled worker can easily find these insects, for example by using fresh meat as bait.

The fly larval extracts of the invention are produced for example by maintaining eggs or larvae of the species Lucilia sericata and/or Sarcophaga carnaria on fresh meat.

The larvae grow and thrive on the meat and are harvested shortly before entry into the pupation stage. It is advantageous in this connection to harvest the larvae in the period from day 5 to day 8 after hatching from the egg.

The larvae are killed and processed about 5 to 8 days after hatching of the larvae from the egg, but before pupation in each case. The killed larvae are cooled before and during the further processing to the fly larval extract. Possible cooling temperatures are temperatures below 0° C., that is to say in the frozen state, for example at temperatures from 0 C. to −80° C. However, it is also possible to work at temperatures from 0° C. to 15° C., preferably from 0° C. to 10° C., in particular from 2° C. to 6° C. The larvae can also be frozen for the homogenization or for further processing or be comminuted and homogenized already in the frozen state.

The larvae are for this purpose initially made substantially sterile externally and freed of any secretions and excretions (SE) which might adhere to the maggot body. This takes place by a plurality of washing steps in aseptic solutions in decreasing concentration. Sterilized NaCl solution is employed in the last washing steps to produce substantial external sterility of the larvae. This also washes off all secretions and excretions of the maggots, and the maggots are preserved on ice.

The larvae are homogenized for example by mechanical comminution or ultrasound. The fly larvae can be homogenized as such or, preferably, with the addition of an extraction medium. From 0.1 ml to 500 ml of extraction solution, preferably 0.5 ml to 100 ml, very preferably 1 to 5 ml, of extraction solution are added per gram wet weight of fly larvae. Sterile extraction solutions are particularly suitable, for example purified water, physiological salt solutions, buffers, electrolyte, sugar or protein solutions and aqueous emulsions, and organic solvents. It is also possible to dispense entirely with addition of extraction media and merely to expel the liquid constituents of the maggots under pressure. The extract can also be produced by precipitating the active substances by addition of organic solvents and subsequently extracting them. Separation of the homogenate into solid and soluble constituents takes place for example by filtration or centrifugation. The fly larval extracts are preserved where appropriate by freezing or by freeze drying. It is also possible to employ other known agents for stabilizing active molecules, for example protease inhibitors, trehalose, ectoin or buffers.

After the homogeneous liquid is obtained, the extract is filtered, for example filtered sterile with a filter which has a pore diameter of from 0.1 μm to 0.4 μm. In the last step, the extract is aliquoted and frozen in liquid nitrogen. Permanent storage takes place at a temperature of about −21° C. to −80° C. or in liquid nitrogen. The resulting extracts which have been sterilized by filtration can also be lyophilized.

The extracts of the invention can also be further purified by conventional purification methods or be fractionated such as by selective precipitation steps or chromatographic or electrophoretic methods.

The invention also relates to pharmaceuticals which have an effective content of the fly larval extracts of the invention together with a pharmaceutically suitable and physiologically tolerated carrier, additive and/or other active substances and excipients.

Because of the pharmacological properties, the fly larval extracts of the invention are suitable for the therapy of superficial or deep chronic and acute wounds of any etiology.

The term “chronic and acute wounds of any etiology” means for example wounds such as surgical wounds which deliberately or unintentionally heal by secondary intention, incision, stab, abrasion, bite, burn or gunshot injuries, and other wounds which cannot primarily be treated by surgical suture or primary wound closure. The term acute wounds also means all wounds which, owing to a superinfection, cannot undergo primary healing and all wounds which have been manifest for 4 weeks and less. Chronic wounds are all injuries associated with abolition of the integrity of the epithelium and are manifest for more than 4 weeks. This means in particular poorly healing wounds based on diabetes mellitus, varicosis or venous thrombosis, a rheumatic disorder, vasculitis, arterial occlusive disease, a disorder of the lymph vessels, hematological disorders and during or after infection of the wounds.

The invention also relates to a process for producing a pharmaceutical, which comprises converting the fly larval extracts of the invention into a suitable dosage form with a pharmaceutically suitable and physiologically tolerated carrier and, where appropriate, other suitable active substances, additives or excipients.

The invention also relates to the use of the fly larval extracts of the invention for producing pharmaceuticals for the therapy of superficial or deep chronic and acute wounds of any etiology.

The pharmaceuticals of the invention are usually applied topically.

Suitable pharmaceutical compositions for topical use on the skin are preferably in the form of a solution, suspension, dusting powder, liposomal formulations, gel, lotion, paste, spray or aerosol. Carriers which can also be used are polyethylene glycols, alcohols and combinations of two or more of these substances. The list can by no means be regarded as restrictive. The fly larval extracts of the invention are present in a concentration of from 0.1% by weight to 100% by weight of the composition, for example from 1.0% by weight to 60% by weight, depending on the extraction conditions.

Transdermal administration is also possible. Suitable pharmaceutical compositions for transdermal uses may be in the form of individual plasters which are suitable for long-term close contact with the patient's epidermis. Plasters of this type suitably contain the fly larval extracts of the invention in an optionally buffered aqueous solution, dissolved and/or dispersed in an adhesive or dispersed in a polymer. A suitable active substance concentration is from about 0.1% by weight to 75% by weight, preferably from 1% by weight to 70% by weight. A special possibility is for the active substance to be released by electrotransport or iontophoresis as described, for example, in Pharmaceutical Research, 2(6): 318 (1986).

The fly larval extracts of the invention can also be applied to the wound through wound coverings made of gauze, of alginates, of hydrocolloid materials, foams and silicone coverings, which have been coated, impregnated or treated with these fly larval extracts and are therefore able to deliver the fly larval extract into or onto the wound or wound surface.

Suitable solid pharmaceutical forms are, for example, granules, powders, solutions, suspensions, emulsions or drops, and products with protracted release of active substance, in the production of which conventional excipients or carriers are used. Excipients which are frequently used and which may be mentioned are magnesium carbonate, titanium dioxide, lactose, mannitol and other sugars, talc, milk protein, gelatin, starch, cellulose and derivatives thereof, polyethylene glycol and solvents such as, for example, sterile water and monohydric or polyhydric alcohols such as glycerol.

The fly larval extracts of the invention may also be employed in pharmaceutical forms which contain the fly larval extracts in inactive form and are then applied into or onto the wound and activated by addition of specific substances. Simple examples are the use of a powder or of lyophilizates which are dissolved with physiological solutions (e.g. 0.9% NaCl). The pharmaceutical preparation may also be a solution if the stability is adequate.

The suitable pharmaceutical compositions are applied after mechanical cleaning of the wound. The mechanical cleaning of the wound takes place for example by a bath or rinsing of the wound with Ringer lactate. The wound is optionally covered after application of the fly larval extracts of the invention by hydrocolloid wound dressings or by contact adhesive surgical film. The dressings are changed each day with new administration of the fly larval extracts of the invention each time.

### EXAMPLE 1

Production of the Fly Larval Extracts of the Invention

Larvae of the species Lucilia sericata and/or Sarcophaga carnaria were maintained on fresh horsemeat with little or no contamination and harvested shortly before entry into the pupation stage. A substantial external sterility of the larvae was produced in several washing steps in aseptic solutions in decreasing concentration and in sterilized NaCl solution in the last washing steps. The larvae were then decapitated, i.e. the front third was divided from the remainder of the body of the larva. Both parts of the larvae were immediately preserved separately in a carrier medium on ice. The larvae were then homogenized. This took place in several steps by mechanical comminution and homogenization using ultrasound. Care was taken to cool continuously to about 4° Celsius. After a homogeneous liquid was obtained, the extract was filtered sterile (Millipore filter). In the last processing step, the extract was aliquoted and frozen in liquid nitrogen. Permanent storage took place at about −21° C. to −80° C.

### EXAMPLE 2

Wound Treatment

2 ml portions of the fly larval extract produced as in Example 1, produced from equal proportions by weight of maggots and physiological saline, were applied to an 82-year old female patient who had suffered from chronic recurrent leg ulcers for some years. The ulcers are of venous origin and were also influenced by intake of analgesics in the sense of vasculitis. At the start of the treatment there were several ulcers, some with fibrinous deposits, on both lower legs. After consultation with the patient, the treatment started with systemic administration of steroids and simultaneous local application of the fly larval extract produced as in Example 1 to promote debridement. Fibrolan® ointment and aqueous solutions of the extract of the invention were employed for comparison. Fibrolan® ointment is a product which is included in the Roten Liste and contains as active substances plasmin from bovine plasma and deoxyribonuclease from bovine pancreas. With the extracts from the larvae, a distinction was made between the extracts from the front part and from the rear part of the larvae. Allocation took place at the start of the therapy and was maintained throughout the treatment. It emerged that occlusive application of the extracts of the invention was distinctly superior to the use of Fibrolan® ointment in relation to the debriding effect and the speed of wound closure. The result of treatment was recorded by means of color photographs.

The fly larval extract produced as in Example 1 was employed for an 87-year old female patient who had suffered from leg ulcers of vasculitic origin for some years. No further ulcerations occurred after systemic intake of steroids. Local therapy compared Fibrolan® ointment and the extracts of the invention. The ulcer which was treated with the extracts of the invention initially had a more pronounced necrotic and fibrinous deposit than the comparative ulcer treated with Fibrolan® ointment. After treatment for 8 days there was seen to be distinctly faster debridement of the ulcer treated with the extracts of the invention compared with the Fibrolan® ointment. The result of treatment was recorded by means of color photographs.

What is claimed is:

1. A process for producing fly larval extract from fly larvae comprising homogenizing the fly larvae and separating the undissolved constituents of the fly larvae from the resulting homogenate.

2. The process of claim 1 wherein the fly larvae are cooled prior to and during homogenization.

3. The process of claim 2 wherein the fly larvae are cooled to below about 15° C.

4. The process of claim 3 wherein the fly larvae are cooled to about 2° C. to about 6° C.

5. The process of claim 2 wherein the homogenization of the fly larvae takes place in the frozen state at temperatures below 0° C.

6. The process of claim 1 wherein an extraction medium is added before the homogenization.

7. The process of claim 6 wherein the extraction medium contains water.

8. The process of claim 6 wherein said extraction medium is selected from the group consisting of water, physiological salt solutions, buffers, electrolyte, sugar or protein solutions, or emulsions.

9. The process of claim 6 wherein the extraction medium is an organic solvent.

10. The process of claim 1 wherein said fly larvae of the genera Sarcophaga, Lucilia, Musca, Calliphora or Stomoxys, or mixtures of representatives of these genera, or from the species Lucilia sericata, Lucilia caesar, Lucilia cuprina, Sarcophaga carnaria, Sarcophaga agyrostoma, Musca domestica, Calliphora erythrocephala, Calliphora vicina or Stomoxys calcitrans.

11. The process of claim 1 wherein the fly larvae are 5 to 8 days old.

12. The process of claim 1 wherein the fly larvae are killed prior to homogenization.

13. The process of claim 1 wherein the homogenization is effected by mechanical comminution or by ultrasound.

14. The process of claim 6 wherein from 0.1 ml to 500 ml of extraction medium is added per gram wet weight of fly larvae.

15 The process of claim 14 wherein 0.5 ml to 100 ml of extraction medium is added per gram wet weight of fly larvae.

16. The process of claim 15 wherein 1 to 5 ml of extraction medium is added per gram wet weight of fly larvae.

17. The process of claim 1 wherein the separation of insoluble constituents in the homogenate takes place by centrifugation or filtration.

18. The process of claim 1 further comprising preserving the fly larval extract by freezing or lyophilization.

19. The process of claim 1 further comprising freeing the fly larvae of discharged secretions and excretions of the larvae by washing before the homogenization.

20. A fly larval extract produced by the process of claim 1.

21. A pharmaceutical composition comprising a pharmaceutically effective amount of a fly larval extract produced by the process of claim 1 together with a pharmaceutically suitable and physiologically tolerated carrier.

22. A pharmaceutical composition in accordance with claim 21 which comprises a pharmaceutically effective amount of fly larval extract for treating wounds.

23. A pharmaceutical composition in accordance with claim 22 which is suitable for topical use on the skin.

24. A pharmaceutical composition in accordance with claim 23 which is in the form of a solution, suspension, dusting powder, cream, liposomal formulation, gel, lotion, paste, spray or aerosol.

25. A pharmaceutical composition in accordance with claim 22 comprising fly larval extract in a concentration of from 0.1% by weight to 100% by weight in the pharmaceutical composition.

26. A wound treatment preparation comprising a plaster for transdermal use containing a pharmaceutical composition in accordance with claim 22.

27. A wound treatment preparation comprising a wound covering made of gauze, alginates, hydrocolloid materials, foams or silicone coverings which are coated or impregnated with a pharmaceutical composition in accordance with claim 22.

28. A method of treating wounds comprising administering a pharmaceutically effective amount of the fly larval extract of claim 20.

29. A method of treating wounds comprising administering a pharmaceutically effective amount of the pharmaceutical composition of claim 22.

30. The method of claim 28 wherein said wounds are superficial.

31. The method of claim 28 wherein said wounds are chronic and acute wounds of any etiology.

32. The method of claim 28 wherein the fly larval extract is provided in a wound treatment preparation in which the fly larval extract is present in inactive form, the preparation is subsequently applied to the wound, and the fly larval extract.
